(19) United States
(12) Patent Application Publication
(10) Pub. No.: US 2025/0224330 A1
(43) Pub. Date: Jul. 10, 2025

(54) UV-BASED, IN-SITU SOIL CARBON MEASUREMENT SYSTEM

(71) Applicant: University of Utah Research Foundation, Salt Lake City, UT (US)

(72) Inventors: Hanseup KIM, Salt Lake City, UT (US); Carlos H. MASTRANGELO, Salt Lake City, UT (US); Steven TRAN, Salt Lake City, UT (US); Ashrafuzzaman BULBUL, Salt Lake City, UT (US); Seungbeom NOH, Salt Lake City, UT (US)

(21) Appl. No.: 18/853,061

(22) PCT Filed: Apr. 27, 2023
(86) PCT No.: PCT/US2023/066318

§ 371 Date: Sep. 30, 2024

Related U.S. Application Data
(60) Provisional application No. 63/335,591, filed on Apr. 27, 2022.

(51) Int. Cl. G01N 21/33; G01N 21/3504; G01N 33/24

(52) U.S. Cl. CPC G01N 21/33; G01N 21/3504; G01N 33/24

(57) ABSTRACT

A soil carbon measurement device (100) can include a subsurface support (110) configured to be inserted at least partially under a soil surface. The subsurface support can include an interior volume (114) and a. gas-permeable barrier separating the interior volume (114) from soil under the soil surface. The device (100) can also include an ultraviolet light source (1.30) positioned to expose the soil under the soil surface to ultraviolet light, and a carbon dioxide sensor (150) configured to measure a concentration of carbon dioxide in the interior volume (114) of the subsurface support (110).

### CROSS REFERENCE TO RELATED APPLICATION

This application claims priority to U.S. Provisional Application No. 63/335,591, filed on Apr. 27, 2022, which is incorporated herein by reference.

### STATEMENT REGARDING FEDERALLY SPONSORED RESEARCH OR DEVELOPMENT

This invention was made with government support under DE-AR0001383 awarded by the U.S. Department of Energy. The government has certain rights in the invention.

### NAMES OF THE PARTIES TO A JOINT RESEARCH AGREEMENT

Not applicable.

### INCORPORATION BY REFERENCE STATEMENT

Not applicable.

### BACKGROUND

As the effects of global climate change concerningly grow each and every year, carbon monitoring and sequestration within the land becomes more important in suppressing said effects. The most common land-use practice for carbon sequestration involves carbon-credits, by which large landowners cooperate with corporations to reduce greenhouse emissions by various means such as sequestrating CO₂ within the land. However, many potential carbon-credit investors are skeptical of introducing agricultural carbon sequestration practices to their land, as measurement uncertainty of sequestration quantity can greatly reduce potential earnings for the investor.

### SUMMARY

This disclosure describes soil carbon measurement devices that can be capable of accurate, in-situ, soil organic carbon (SOC) measurements within a localized area of soil at near real-time. In one example, a soil carbon measurement device can include a subsurface support configured to be inserted at least partially under a soil surface. The subsurface support can include an interior volume and a gas-permeable barrier separating the interior volume from soil under the soil surface. An ultraviolet light source can be positioned to expose the soil under the soil surface to ultraviolet light. The device can also include a carbon dioxide sensor configured to measure a concentration of carbon dioxide in the interior volume of the subsurface support.

In another example, a method of measuring soil carbon can include inserting a soil carbon measurement device at least partially under a soil surface. The soil carbon measurement device can include an interior volume and a gas-permeable barrier separating the interior volume from the soil under the soil surface. The method can also include exposing the soil under the soil surface to ultraviolet light and measuring a concentration of carbon dioxide in the interior volume of the soil carbon measurement device.

In another example, a soil carbon monitoring system can include an array of soil carbon measurement devices distributed over an area of land. The individual soil carbon measurement devices can include a subsurface support inserted at least partially under a soil surface. The subsurface support can include an interior volume and a gas-permeable barrier separating the interior volume from soil under the soil surface. The devices can also include an ultraviolet light source positioned to expose the soil under the soil surface to ultraviolet light, a carbon dioxide sensor configured to measure a concentration of carbon dioxide in the interior volume of the subsurface support, and a wireless transmitter configured to transmit the measured concentration of carbon dioxide. The system can also include a monitoring station comprising a wireless receiver configured to the receive measured concentrations of carbon dioxide from the soil carbon measurement devices.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a front view of an example soil carbon measurement device in accordance with the present disclosure.

FIG. 2 is a side cross-sectional view of an example soil carbon measurement device in accordance with the present disclosure.

FIG. 3 is a front view of another example soil carbon measurement device in accordance with the present disclosure.

FIG. 4 is a flowchart illustrating an example method of measuring soil carbon in accordance with the present disclosure.

FIG. 5 is a schematic illustration of diffusion carbon dioxide into an example soil carbon measurement device in accordance with the present disclosure.

FIG. 6 is a schematic illustration of an example soil carbon measurement system in accordance with the present disclosure.

FIG. 7 is a graph of measured CO₂ concentration in ppm vs. time in hours in accordance with one example of the present disclosure.

FIG. 8 is another graph of measured CO₂ concentration in ppm vs. time in hours in accordance with another example of the present disclosure.

FIG. 9 is a three-dimensional graph of photo-oxidized CO₂ vs. bulk soil density in g/cm³ vs. soil organic carbon in percent in accordance with still another example of the present disclosure.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “an interior volume” includes reference to one or more of such spaces and reference to “the sensor” refers to one or more of such devices.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Soil Carbon Measurement Devices

A variety of processes have been used for measuring soil carbon content. However, many soil carbon measurement methodologies have low precision, utilize non-in-situ measurements, or require power and time intensive procedures to quantify the amount of carbon within a soil sample. Indirect soil carbon measurements, such as infrared spectroscopy or satellite imaging, may excel in rapid soil analyzation but these methods can be limited by post-processing requirements, environmental condition susceptibility, and soil surface scope of measurements. Direct soil carbon measurements, including wet-oxidation and soil burning techniques, are “gold standards” for soil organic carbon quantification, however, these techniques require time intensive sampling and transferring of soil samples to a laboratory in which a high-powered analysis mechanism is utilized. This precludes quick, localized measurements.

Soil carbon measurement in remote locations can also present challenges due at least in part to lack of utility power lines and limited battery lifetimes. In some applications a large number (i.e. hundreds or thousands) of sensors may be desirable for monitoring soil carbon content. Battery replacement schedules for such systems would be unrealistic in some circumstances. Furthermore, remote locations can be difficult to access, e.g. rural farms, forests, etc.

The soil carbon measurement devices described herein can provide in-situ soil carbon measurements from any desired soil location. To enable this functionality, an ultraviolet light source such as a UV light emitting diode (LED) can be included in the device and placed at a desired soil depth. When soil is exposed to UV wavelengths, the UV radiation induces a photo-oxidation reaction in which the UV provides sufficient energy to generate near-instantaneous carbon dioxide (CO₂) from aromatic carbon compounds within the soil. The aromatic carbon compounds can include dissolved organic carbon (DOC). The CO₂ produced by photo-oxidation is then measured using a CO₂ sensor. The measured concentration of CO₂ can be wirelessly transferred to a remote user. The concentration data can be extrapolated to accurately predict the soil organic carbon levels. This can obviate the time consuming and destructive soil sampling methods (e.g., digging, pumping) utilized in state-of-the-art techniques. Experimental tests of the soil carbon measurement devices described herein in various types of soil around the United States has shown consistent and accurate predictions of soil organic carbon levels when compared to the United States Department of Agriculture's (USDA) national soil survey, with an average percent error of 2.64%, showing potential as a SOC measuring alternative that can help elevate soil and CO₂ sequestration research.

The soil carbon measurement devices described herein can consume very little energy. In some examples, the devices can operate at a power level of about 3 W, in some cases from 0.01 to 10 W, from 0.01 to 5 W, and in other cases from 0.5 to 5 W. Some lab-based UV photo-oxidation processes use significantly higher levels, such as greater than 100 W. Other methods, such as in-lab oxidation and combustion of soil samples can use much more energy, such as multiple kW of power. The soil carbon measurements devices described herein can utilize such a small amount of power, in part, because the devices can operate at ambient temperature. Other methods can utilize high temperatures, such as dry combustion at temperatures around 950° C. The soil carbon measurement devices described herein can also be programmed to operate in low-power “sleep mode” when measurements are not being taken, and the devices can be wirelessly activated in order to make measurements. When the device is in “awake” mode, the only components consuming power may include a UV light source such as a micro-UV LED, a carbon dioxide sensor, and a microcontroller with a wireless transmitter to transmit data to a remote user. In some examples, the soil carbon measurement device can include a battery that can allow for more than a year of periodic soil carbon monitoring.

The soil carbon measurement devices described herein can directly measure soil carbon content, but in a non-invasive way without digging or sampling the soil. The devices can also provide on-the-spot real-time measurements. Other non-invasive methods have been used to measure soil organic carbon content. However, these other methods are indirect methods, such as infrared spectroscopic methods, imaging methods, gamma radiation detection, and eddy covariance. The soil carbon measurement devices described herein can provide higher precision compared to these indirect methods. The soil carbon measurement devices can also be used to measure soil carbon content at different depths beneath the surface of the soil, which is not possible with aerial or satellite-based measurement methods.

Soil can often have a natural CO₂ content due to respiration of microbes in the soil. The soil carbon measurement devices described herein can be used to distinguish between CO₂ produced by microbial respiration and CO₂ produced by photo-oxidation using the UV light source. In some examples, a measurement of the soil CO₂ concentration can be recorded while the UV light source is turned off. This can represent the CO₂ level in the soil due to microbial respiration. The UV light source can then be turned on. The UV light can immediately produce CO₂ by photo-oxidation of organic carbon compounds in the soil. After waiting for a sufficient time to allow the CO₂ to diffuse from the soil into the soil carbon measurement device, another measurement can be recorded. This second measurement can show a higher CO₂ concentration than the first measurement, because the second measurement includes both CO₂ from microbial respiration and CO₂ from photo-oxidation. These measurements can provide useful information about sequestration of carbon in the soil, such as by showing the amount of carbon compounds that are maintained in the soil without being broken down by microbial respiration. As a general rule photo-oxidation effects can be measured within 30 minutes, and in some cases within 20 minutes, of exposure to UV light or cessation of such exposure.

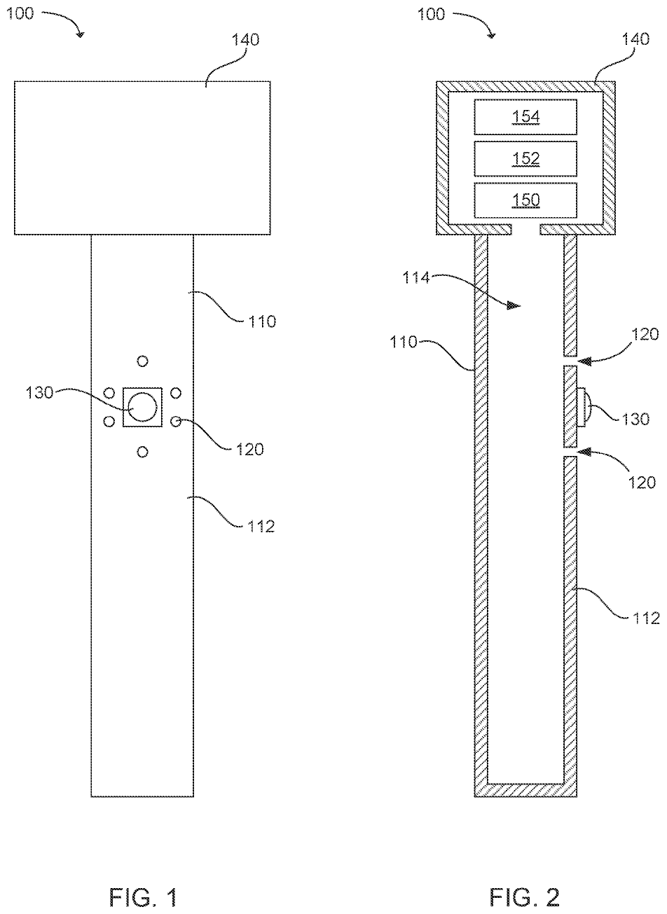

FIG. 1 shows a front view of an example soil carbon measurement device 100. This device includes a subsurface support 110 that can be inserted into the ground so that at least a part of the subsurface support is below the soil surface. The subsurface support can include an interior volume and a gas-permeable barrier separating the interior volume from the soil. For example, the subsurface support can be a hollow tube or other hollow structure. In one example, the hollow of the subsurface support can be at least partially filled with a highly porous matrix material (i.e. porous sponge, fibrous matrix, or other gas permeable matrix). In these cases, the UV light source can be oriented to expose soil to UV light. In one example, the gas permeable barrier is the outer wall 112 of the subsurface support, which has holes 120 to allow gas to pass through the wall. The device also includes an ultraviolet light source 130 positioned to expose the soil under the soil surface to ultraviolet light. In this example, the ultraviolet light source is a UV-LED. The UV-LED is affixed to the outside of the subsurface support. The holes in the wall of the subsurface support can be located near the UV-LED so that carbon dioxide formed by photooxidation in the soil can quickly diffuse through the soil, to the holes, and into the interior volume of the subsurface support. The example shown in FIG. 1 also includes an above-ground enclosure 140 connected to the subsurface support. The above-ground enclosure can contain a carbon dioxide sensor (not shown) that is configured to measure the concentration of carbon dioxide in the interior volume of the subsurface support. In various examples, the carbon dioxide sensor can be inside the above-ground enclosure, or inside the subsurface support, or in another suitable location so long as the carbon dioxide sensor can measure the concentration of carbon dioxide in the interior volume of the subsurface support. In certain examples, the carbon dioxide sensor can be connected to the interior volume of the subsurface support by a diffusion pathway that can allow carbon dioxide to diffuse to the carbon dioxide sensor. This example device can operate by using the UV-LED to expose subsurface soil to ultraviolet radiation. This can produce a photo-oxidation that converts carbon compounds in the soil to CO₂. The CO₂ can then diffuse through the soil to the holes in the subsurface support, and then diffuse through the holes into the interior volume of the subsurface support. The carbon dioxide sensor can then be used to measure the carbon dioxide concentration in the interior volume.

FIG. 2 shows a cross-sectional schematic view of the example soil carbon measurement device 100. In this view, the hollow interior volume 114 of the subsurface support 110 can be seen. The interior volume is surrounded by the outer wall 112 of the subsurface support. The holes 120 penetrate through the outer wall so that gas can diffuse from the soil to the interior volume. The ultraviolet light source 130 is positioned on the outer surface of the outer wall and configured to expose soil to ultraviolet light. The above-ground enclosure 140 is connected to the top of the subsurface support. In this example, the above-ground enclosure has an opening 120 into the interior volume of the subsurface support. A carbon dioxide sensor 150, battery 152, and wireless transmitter 154 are inside the above-ground enclosure. In some examples, a microcontroller can be included. The carbon dioxide sensor, battery, and/or wireless transmitter can be integrated parts of the microcontroller or separate components connected to the microcontroller.

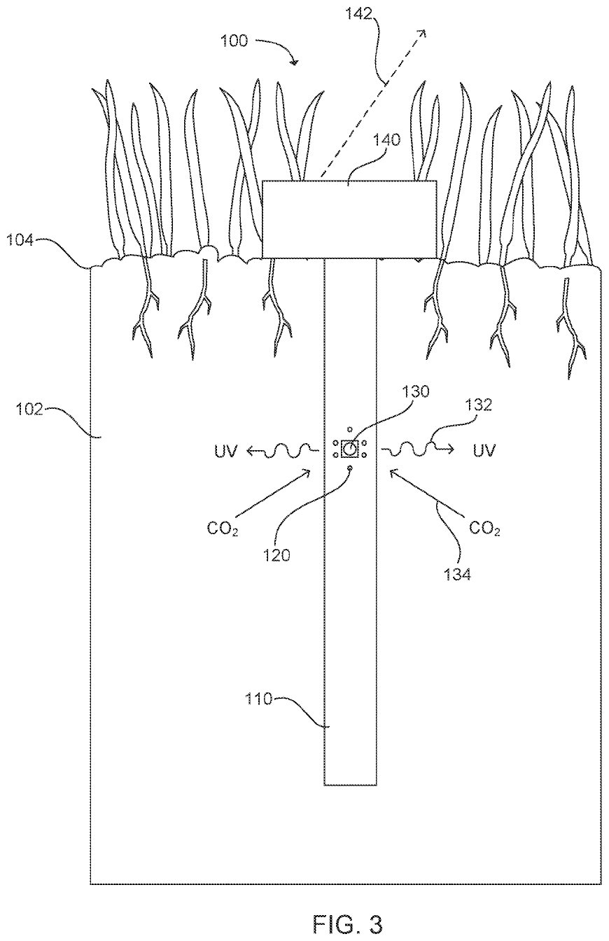

FIG. 3 shows a side view of an example soil carbon measurement device 100 inserted into soil 102 to illustrate the operation of the device. The subsurface support 110 is inserted under the soil surface 104. The length of the subsurface support and depth at which the ultraviolet light source 130 is located can vary depending on the desired depth for measuring soil organic carbon content. The ultraviolet light source produces ultraviolet radiation 132 that penetrates a distance into the soil around the ultraviolet light source. The ultraviolet radiation provides energy to drive chemical reactions resulting the oxidation of carbon compounds in the soil, forming carbon dioxide 134. The carbon dioxide then diffuses to the holes 120 and into the interior volume of the subsurface support. After a sufficient time for diffusion, the carbon dioxide can be measured by the carbon dioxide sensor. As in the previous example, the carbon dioxide sensor can be inside the above-surface enclosure 140. When the device is inserted into the soil, the above-surface enclosure can remain above the soil surface. A wireless transmitter can also be included in the above-surface enclosure to transmit a wireless signal 142 comprising the measured carbon dioxide concentrations.

In more detail regarding the soil carbon measurement devices, the subsurface support can have an elongated shape that allows the subsurface support to extend downward beneath the surface of soil. The subsurface support can be shaped as a shaft or shank. In some examples, the subsurface support can have a tubular shape with a cross section such as a circular, square, rectangular, or other cross section. In certain examples, the subsurface support can be formed from a cylindrical pipe or tube. In further examples, the subsurface support can have an open end, a closed end, a flat end, a tapered end, a rounded end, or another shape at the end of the support. In some cases, a rounded, tapered, or pointed end can be useful to allow the subsurface support to be driven into the soil.

The subsurface support can be made from any suitable material. In some examples, the subsurface support can be made from a rigid material such as metal or plastic. Specific examples can include steel, iron, aluminum, copper, brass, polyvinyl chloride, acrylonitrile butadiene styrene, polypropylene, polyurethane, or others. The subsurface support can have an outer wall with a wall thickness from about 1 mm to about 1 cm, or from about 1 mm to about 5 mm, or from about 1 mm to about 3 mm. The diameter of the subsurface support is not particularly limited. In some examples, the diameter of the subsurface support can be from about 1 cm to about 5 cm, or from about 1 cm to about 3 cm, or from about 2 cm to about 4 cm.

The length of the subsurface support can be selected to allow for measurement of soil organic carbon content at a particular depth in the soil. In some examples, the subsurface support can have a length from about 5 cm to about 100 cm, or from about 20 cm to about 100 cm, or from about 40 cm to about 100 cm, or from about 60 cm to about 100 cm, or from about 25 cm to about 75 cm, or from about 50 cm to about 75 cm, or from about 60 cm to about 75 cm.

In certain examples, the subsurface support can have an interior volume from about 1 mL to about 2 L, or from about 1 mL to about 1 L, or from about 10 mL to about 1 L, or from about 50 mL to about 1 L, or from about 50 mL to about 500 mL. The volume of the interior volume can affect the concentrations of carbon dioxide that may be measured in the interior volume. For example, in a larger interior volume, the same amount of carbon dioxide produced from the soil will cause a smaller concentration change as the carbon dioxide spreads throughout the larger volume. The soil carbon measurement devices can be calibrated against soil samples with known SOC content so that the SOC content of other soils can be accurately extrapolated from measured concentrations of carbon dioxide.

The subsurface support can include a gas-permeable barrier that allows carbon dioxide to diffuse from the soil into the interior volume of the subsurface support. In some examples, the gas-permeable barrier can comprise holes formed in the subsurface support. The holes can be drilled or formed in any other suitable way to provide a diffusion pathway for gas to diffuse from the soil into the interior volume. In some examples, the holes can be small enough in size to prevent most soil particles from entering the interior volume through the holes. However, even if some soil enters the interior volume through the holes, carbon dioxide can still diffuse through the soil and into the interior volume of the subsurface support. Therefore, the holes may not exclude all soil in some examples. In some examples, the holes can have a width from about 10 micrometers to about 5 millimeters, or from about 100 micrometers to about 3 millimeters, or from about 500 micrometers to about 3 millimeters, or from about 1 mm to about 3 mm. The holes can be formed in the subsurface support at a location where it is desired to measure soil carbon content. In some examples, the holes can be positioned at a depth from about 5 cm beneath the soil surface to about 100 cm beneath the soil surface, or from about 5 cm to about 80 cm beneath the soil surface, or from about 15 cm to about 80 cm beneath the soil surface, or from about 25 cm to about 75 cm beneath the soil surface, or from about 50 cm to about 75 cm beneath the soil surface, or from about 60 cm to about 75 cm beneath the soil surface. In further examples, the holes can be located near an ultraviolet light source. In certain examples, the holes can be arranged encircling the ultraviolet light source. For example, the ultraviolet light source can be encircled by a number of holes. The number of holes around a single ultraviolet light source can be from 1 to 20 in some examples, or from 2 to 20, or 4 to 20, or 6 to 20, or 10 to 20, or 2 to 16, or 2 to 12, or 2 to 10 in other examples. The holes can be located near the ultraviolet light source so that carbon dioxide formed by photo-oxidation near the ultraviolet light source can diffuse quickly into the holes. In some examples, at least one hole can be within a distance from about 1 mm to about 2 cm from the ultraviolet light source. In other examples, at least one hole can be from about 1 mm to about 1 cm, or from about 1 mm to about 5 mm, or from about 5 mm to about 1 cm away from the ultraviolet light source.

Other types of gas-permeable barriers can also be used. In other examples, the gas-permeable barrier can include a gas-permeable membrane, a mesh, a screen, or other barrier. The gas-permeable barrier can prevent most soil from entering into the interior volume of the subsurface support while allowing gases to diffuse from the soil into the interior volume. In some examples, the entire subsurface support can be made from a porous material such as a membrane, a mesh, or a screen. In other examples, a portion of the subsurface support can be made of a porous material and the remainder of the subsurface support can have solid walls.

The soil carbon measurement devices can also include an ultraviolet light source. The ultraviolet light source can be positioned to expose the soil under the soil surface to ultraviolet light. In some examples, the ultraviolet light source can be positioned along the subsurface support. For example, the ultraviolet light source can be attached to an outer surface of the subsurface support, or embedded in the wall of the subsurface support, or placed in the interior volume of the subsurface support if the subsurface support has an opening or transparent portion to allow ultraviolet light to reach the soil. The ultraviolet light source can penetrate a small distance into the soil. For example, the ultraviolet light may penetrate from 1 mm to 5 cm, or from 1 mm to 3 cm, or from 1 mm to 1 cm into the soil around the ultraviolet light source. Therefore, the ultraviolet light can trigger chemical reactions that convert carbon compounds in this surrounding soil into CO₂. The ultraviolet light source can be positioned along the subsurface support at a depth where it is desired to measure the soil organic carbon content. In some examples, the ultraviolet light source can be positioned from about 1 cm to about 95 cm below the soil surface when the subsurface support is inserted under the soil surface. In further examples, the ultraviolet light source can be positioned from about 5 cm to about 80 cm, or from about 10 cm to about 80 cm, or from about 25 cm to about 75 cm, or from about 50 cm to about 75 cm, or from about 60 cm to about 75 cm below the soil surface.

In the examples shown in the figures above, the soil carbon measurement device includes a single ultraviolet light source. A single ultraviolet light source can be sufficient to measure soil organic carbon content at the depth where the single ultraviolet light source is positioned. In other examples, the soil carbon measurement device can include multiple ultraviolet light sources. The multiple ultraviolet light sources can be arranged along the subsurface support at different depths or at a single depth. In some examples, the multiple ultraviolet light sources can face in different directions or in a single direction. In some cases, multiple light sources at different depths can be activated simultaneously to generate photo-oxidized CO₂ from multiple different depths in the soil. The measured concentration of CO₂ can then be used to extrapolate an average soil organic carbon content across the different depths in the soil. Alternatively, the multiple light sources can be activated individually to measure the soil organic carbon content at different depths in the soil. Individual measurements can be taken at each of the different depths and this can provide information about how the carbon content varies with depth in the soil. When multiple ultraviolet light sources are used, the subsurface support can include gas permeable portions, such as holes, near each of the ultraviolet light sources so that photo-oxidized carbon dioxide can diffuse into the interior volume of the subsurface support near each ultraviolet light source.

The ultraviolet light source can emit electromagnetic radiation having a wavelength from about 100 nm to about 450 nm. In some examples, the wavelength can be from 315 nm to 450 nm, or from 400 nm to 450, or from 400 nm to 425 nm. The ultraviolet light source can emit other wavelengths in addition to the wavelengths described above, as long as the ultraviolet light source emits a wavelength with sufficient energy to cause photo-oxidation of organic carbon compounds. In some examples, the ultraviolet light source can be a UV-LED. Specific examples can include 365 nm LEDs, 385 nm LEDs, 395 nm LEDs, 405 nm LEDs, 415 nm LEDs, and others. The UV-LED can be a micro-LED that consumes a small amount of power. In some examples, the UV-LED can consume from about 500 mW to about 10 W, or from about 1 W to about 5 W, or from about 1 W to about 3 W of power. Other types of ultraviolet light sources can also be used, such as ultraviolet incandescent bulbs, low pressure mercury lamps, and ultraviolet fluorescent or compact fluorescent lamps.

The carbon dioxide sensor can measure the concentration of carbon dioxide gas in the air within the interior volume of the subsurface support. In some examples, the carbon dioxide sensor can be configured to measure carbon dioxide concentrations from about 100 ppm to about 1,000 ppm. The carbon dioxide sensor can be a nondispersive infrared (NDIR) carbon dioxide sensor in some examples. NDIR sensors can include single beam and dual beam NDIR sensor. In other examples, the carbon dioxide sensor can be a photo-acoustic sensor, an electrochemical sensor, a metal oxide semiconductor sensor, or another type of carbon dioxide sensor. The carbon dioxide sensor can be connected to a microcontroller or integrated as a part of a microcontroller in some examples.

In various examples, the carbon dioxide sensor can be located anywhere that allows the sensor to measure the carbon dioxide concentration of gas in the interior volume of the subsurface support. In some examples, the carbon dioxide sensor can be located inside the subsurface support so that the carbon dioxide sensor can be directly exposed to the gas in the interior volume. In further examples, the carbon dioxide sensor can be located in an above-ground enclosure. The above-ground enclosure can have an opening that allows gas from the interior volume of the subsurface support to diffuse into the above-ground enclosure. Thus, the carbon dioxide sensor can measure the concentration of carbon dioxide in the gas as it diffuses from the interior volume of the subsurface support into the above-ground enclosure. In still further examples, a sample of gas from the interior volume can be introduced to the carbon dioxide sensor through a tube or line that leads from the interior volume of the subsurface support to the carbon dioxide sensor. The gas sample can travel through the tube or line by passive diffusion or a pump or blower can be used to cause the gas to travel to the carbon dioxide sensor.

The soil carbon measurement device can also include a microcontroller. The microcontroller can be connected to the carbon dioxide sensor, the ultraviolet light source, a wireless communication module, and any other components of the device that are to be electronically controlled. The microcontroller can include a processor, memory, and/or other electronic components. Specific examples of microcontrollers that can be used include TEENSY® microcontrollers available from PJRC (Unites States).

The microcontroller can be connected a wireless communication module in some examples. A wireless communications module that includes a wireless transmitter can be used to transmit the measured carbon dioxide concentration data to a remote user or base station. A wireless communications module that can also receive wireless signals can be useful to allow a remote user or base station to transmit instructions to the soil carbon measurement device. In some examples, the wireless communication module can be configured to communicate by radio, Wi-Fi, Bluetooth, cellular network, satellite, or other wireless communication methods. In certain examples, the wireless communication module can be a LORA™ transceiver available from LORA ALLIANCE® (United States).

The soil carbon measurement device can also include a battery or other power source to supply power the ultraviolet light source, carbon dioxide sensor, microcontroller, and wireless communication module. In some examples, the battery can be charged with sufficient energy to run the soil carbon measurement device for 1 month to 5 years, or from 2 months to 2 years, or from 6 months to 2 years, or from 6 months to 1 year without recharging. During this operating time, the device can be programmed to sleep in a low-power sleep mode for a majority of the time and wake up periodically to take soil carbon measurements at certain intervals. In some examples, an individual soil carbon measurement device in sleep mode can draw standby power from 1 mW to 100 mW, or from 5 mW to 50 mW, or from 10 mW to 25 mW. Other power sources that can be used to power the soil carbon measurement device include wired power connections, solar panels, wind-powered generators, and others.

### Methods of Measuring Soil Carbon

The present disclosure also described methods of measuring soil carbon content. These methods can be performed using the soil carbon measurement devices described above. Additionally, in some examples the methods can be implemented by a computer or multiple computers. In particular, the soil carbon measurement devices can include controllers as described above, and the controllers can include one or more processors. The soil carbon measurement devices can also be in communication with one or more additional computers, such as a base station computer. The additional computers can also include processors. The processors of the soil carbon measurement devices and other computers with which they communicate can be used to perform the methods described herein.

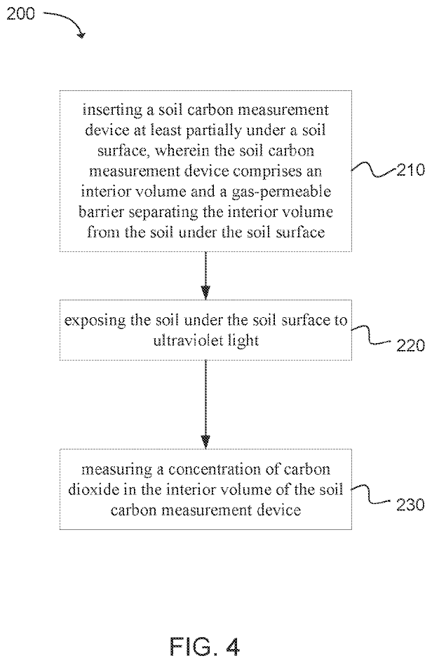

FIG. 4 is a flowchart illustrating an example method 200 of measuring soil carbon. This method includes: inserting a soil carbon measurement device at least partially under a soil surface, wherein the soil carbon measurement device comprises an interior volume and a gas-permeable barrier separating the interior volume from the soil under the soil surface 210, exposing the soil under the soil surface to ultraviolet light 220; and measuring a concentration of carbon dioxide in the interior volume of the soil carbon measurement device 230.

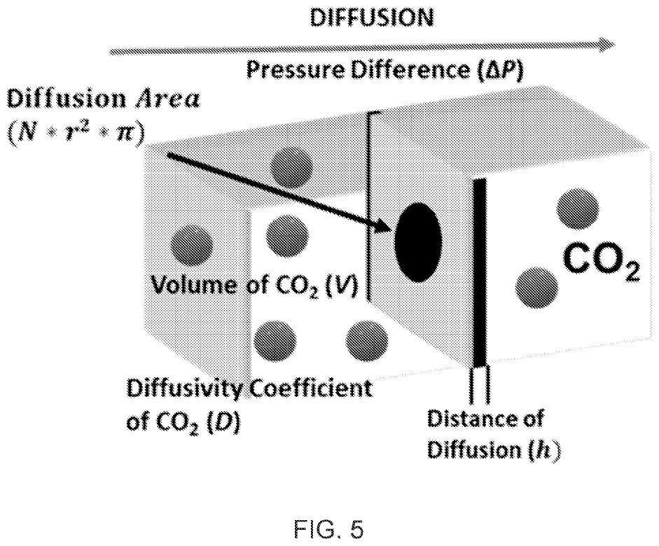

FIG. 5 shows a schematic example of how carbon dioxide molecules diffuse from the soil through holes in the subsurface support of the soil carbon measurement device. The rate of diffusion is based on Fick's law of diffusion. A time constant “τ” for the diffusion rate of CO₂ can be derived utilizing the pressure “P” at the interface of the diffusion holes, the volume of CO₂ “V”, the CO₂ diffusivity coefficient “D”, the diffusion distance “h”, time “t”, and the diffusion area, as illustrated in FIG. 5. The time constant can be found using the following formula:

$$τ = \frac{Vht}{{\int }_{0}^{t} Δ {PNr}^{2} π D}$$

As mentioned above, microbes in the soil can produce carbon dioxide through respiration. Therefore, there can be a background CO₂ concentration in the soil due to microbial respiration, before the ultraviolet light is ever applied to the soil. When the soil is exposed to ultraviolet light, additional CO₂ can be formed by photo-oxidation. Without being limitation to a particular mechanism, in some examples the energy provided by the ultraviolet radiation can be transferred to O₂ molecules to create singlet oxygen which then reacts with surrounding compounds within the soil to form H₂O₂. The H₂O₂ can then go through a Fenton reaction, producing reactive oxygen species in the form of a hydroxyl radical. These hydroxyl radicals can then react with the aromatic carbons found in the dissolved organic carbon components of soil to generate photo-oxidized CO₂. Differentiation of photo-oxidized and microbial-respirated CO₂ sources can be achieved through dependence on pressure, where the UV generated photo-oxidized CO₂ causes a higher pressure on the outer interface, causing an influx of photo-oxidized CO₂ to diffuse into the interior volume of the device for sensing.

In some examples, the background carbon dioxide generated by microbial respiration can be differentiated from carbon dioxide generated by photo-oxidation by measuring the carbon dioxide for a first time period while the ultraviolet light source is off, and then measuring the carbon dioxide for a second time period while the ultraviolet light source is on. During the first time period, the carbon dioxide sensor can often show a relatively steady state value of the carbon dioxide present in the soil due to microbial respiration. When the ultraviolet light source is turned on, additional carbon dioxide is immediately formed by photo-oxidation. However, it can take some time for the photo-oxidized carbon dioxide to diffuse into the soil carbon measurement device. The readings of the carbon dioxide sensor can often show a sudden increase in carbon dioxide concentration during the first few minutes after turning on the ultraviolet light source. After a sufficient time for diffusion has passed, the carbon dioxide concentration readings can settle to a new steady state concentration. This concentration represents the carbon dioxide generated by microbial respiration plus the carbon dioxide generated by photo-oxidation. The concentration during the first time period can be compared to the concentration during the second time period to estimate the amount of dissolved organic carbon in the soil. In further examples, it can also be useful to measure the carbon dioxide concentration during a third time period after turning the ultraviolet light source off. After the ultraviolet light source is turned off, the carbon dioxide concentration can fall back to the original level of carbon dioxide generated by microbial respiration alone. In certain examples, the first period of time can be from about 20 minutes to about 2 hours, or from about 1 hour to about 1.5 hours. The second time period can be from about 10 minutes to about 1 hour, or from about 15 minutes to about 45 minutes. The third time period can be from about 20 minutes to about 2 hours, or from about 1 hour to about 1.5 hours.

The methods can also include waking up the soil carbon measurement device from a low-power sleep state, as explained above. In some cases, the device can be awoken by a wireless signal transmitted to the device from a remote user or base station. In other cases, the device can include a wake-up circuit that automatically wakes the device up after a set time interval and then puts the device back to sleep after taking carbon measurements.

### Soil Carbon Monitoring Systems

The soil carbon measurement devices can also be used to make larger soil carbon monitoring systems which include multiple measurement points and devices as a networked array. For example, multiple soil carbon measurement devices can be combined in an array over an area of land. The soil carbon measurement devices can record carbon measurements at different locations across the area of land and a map of the land can be generated showing the soil carbon content in the different locations. In some examples, the soil carbon measurement devices can be configured to measure carbon content at different depths in the soil. For example, as described above, a soil carbon measurement device can have multiple ultraviolet light sources positioned at different depths to measure carbon content at the different depths. By using an array of such soil carbon measurement devices, a three-dimensional map of the soil carbon content can be generated. The three-dimensional map can show the soil carbon content at different geographic locations and at different depths in the soil.

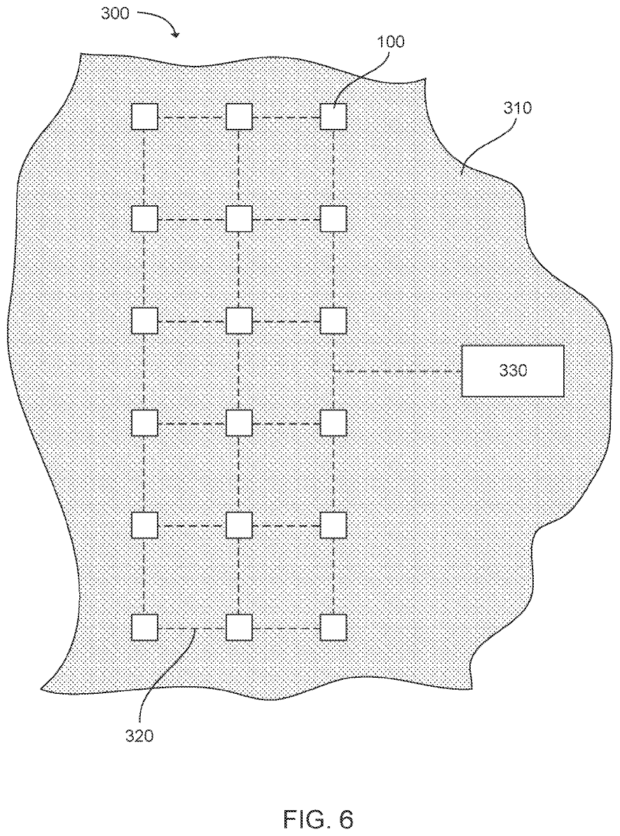

FIG. 6 shows one example soil carbon monitoring system 300. The system includes an array of soil carbon measurement devices 100 distributed over an area of land 310. The individual soil carbon measurement devices can include any of the components described above. In this example, the soil carbon measurement devices can include wireless transmitters that allow the devices to transmit carbon dioxide concentration measurements through a wireless connection 320 (represented by dashed lines) to a monitoring station 330.

### Examples

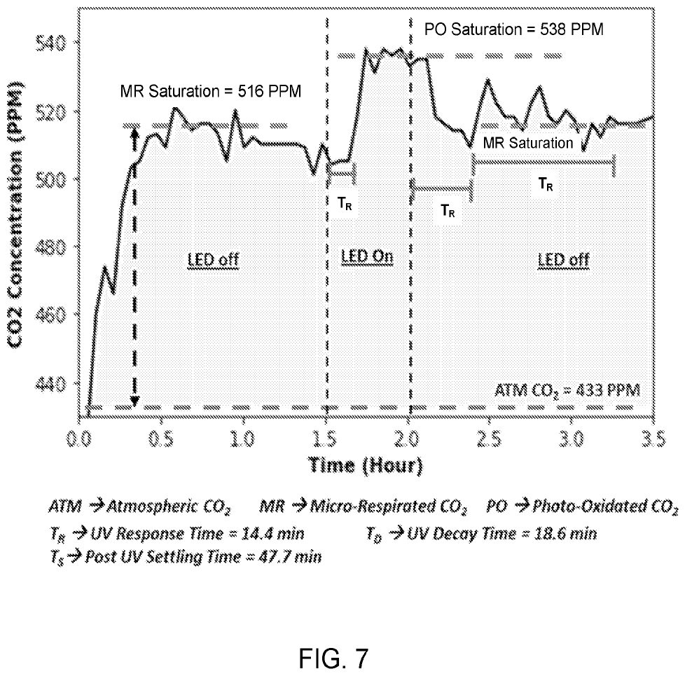

An example soil carbon measurement device was constructed having the basic design shown in FIG. 1. The device had a tube-shaped subsurface support with a micro-UV LED attached to the outside of the subsurface support. The LED output a wavelength of 415 nm and provided 3 eV of energy to the soil. The device also had an above-ground enclosure containing a microcontroller connected to a NDIR carbon dioxide sensor and a LORA™ wireless communication module available from LORA ALLIANCE® (United States). The subsurface support was inserted into the soil and then the carbon dioxide sensor was used to record the carbon dioxide concentration for a period of 90 minutes with the UV LED turned off. The UV LED was then turned on and the carbon dioxide concentration was recorded for a period of 30 minutes, followed by another period of 90 minutes with the UV LED turned off. Initial testing within a Salt Lake City, Utah soil showed that the base-line CO₂ concentration saturated at 512 ppm due to 83 ppm increase from the atmospheric CO₂ level within the first 90-minute UV off time period. When the UV LED was activated, the CO₂ concentration saturated at 538 ppm in 14.4 minutes with 29 ppm of photo-oxidative CO₂ produced within the 30-minute UV on time period. The CO₂ concentration then dropped back down to the base-line CO₂ level after the UV LED was deactivated for roughly 18.62 minutes. The concentration of CO₂ in ppm is shown in FIG. 7. This result shows that the UV based soil carbon measuring system can (1) produce CO₂ during the UV on period for near real-time SOC prediction and (2) photo-oxidized and microbial-respirated CO₂ can be differentiated for CO₂ monitoring.

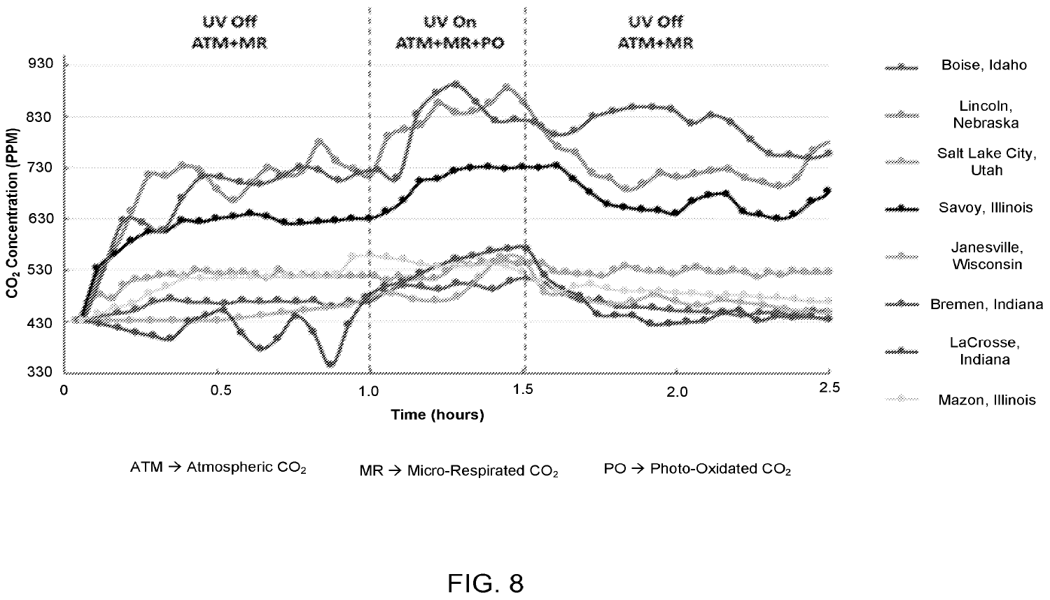

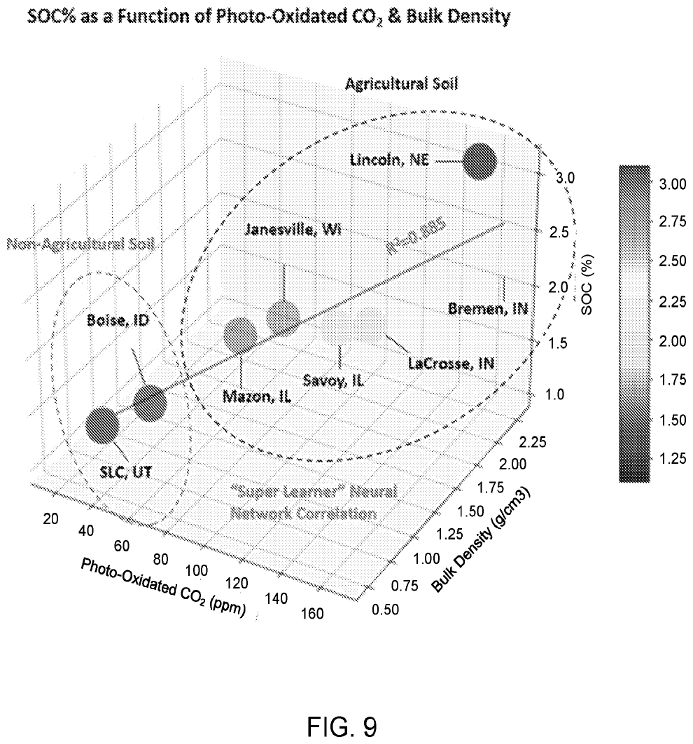

The soil carbon measurement device was additionally tested in eight different locations (Boise, Idaho; Lincoln, Nebraska; Salt Lake City, Utah; Savoy, Illinois; Mazon, Illinois; Janesville, Wisconsin; Bremen, Indiana; and LaCrosse, Indiana) to identify its consistency and accuracy in prediction of SOC. The results of these field tests are illustrated in FIG. 8 and FIG. 9, where the data was normalized to the same atmospheric CO₂ level at 430 ppm. For this test, the UV off period was 60 minutes instead of 90, but the baseline steady states were still achieved within the given period of time. The four tested sites can be categorized in to either non-agricultural soil (Boise, Idaho & Salt Lake City, Utah) or agricultural soil (Lincoln, Nebraska; Savoy, Illinois; Mazon, Illinois; Janesville, Wisconsin; Bremen, Indiana; & LaCrosse, Indiana) based on the size and active crop production of each soil. The field-testing results showed that 39 ppm, 265 ppm, 95 ppm, 203 ppm, 7 ppm, 276 ppm, 3 ppm, and 86 ppm of microbial-respirated was observed in Boise, Lincoln, Salt Lake City, Savoy, Janesville, Bremen, LaCrosse, and Mazon respectively. When the UV LED was activated, 39 ppm, 154 ppm, 30 ppm, 111 ppm, 57 ppm, 167 ppm, 123 ppm, and 56 ppm of photo-oxidized CO₂ was observed each of the eight respective test sites. It can be seen that a noticeably higher CO₂ level was observed by the agricultural soil in comparison to the non-agricultural soil by a factor of 2.74×. These field tests show (1) the consistency of achieving the steady state CO₂ levels during the UV on and off period and (2) that there is a difference in captured CO₂ based on the different soil types leading to different SOC prediction.

The photo-oxidized CO₂ captured by the soil organic carbon measurements can be linearly correlated to the SOC. Results show that the photo-oxidized CO₂ correlates very well with the SOC levels from data measured using “gold standard” methods such as soil combustion, and with a neural network correlation model, with a correlation coefficient of R²=0.885 as shown in FIG. 9. This linear prediction algorithm resulted in a total average error of 9.6%. This data confirms the viability of utilizing the UV-based soil carbon measurement devices to achieve in-situ, near real-time SOC prediction that is on par in accuracy with other soil organic carbon measurement techniques.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, a non-transitory machine readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A soil carbon measurement device, comprising:
a subsurface support configured to be inserted at least partially under a soil surface, wherein the subsurface support comprises an interior volume and a gas-permeable barrier separating the interior volume from soil under the soil surface;
an ultraviolet light source positioned to expose the soil under the soil surface to ultraviolet light; and
a carbon dioxide sensor configured to measure a concentration of carbon dioxide in the interior volume of the subsurface support.

2. The soil carbon measurement device of claim 1, wherein the subsurface support has a length from about 5 cm to about 100 cm.

3. The soil carbon measurement device of claim 1, wherein the subsurface support has a hollow tube shape.

4. The soil carbon measurement device of claim 1, wherein the gas-permeable barrier comprises holes formed in the subsurface support.

5. The soil carbon measurement device of claim 4, wherein the holes have a width from about 10 micrometers to about 5 millimeters.

6. The soil carbon measurement device of claim 1, wherein the ultraviolet light source is positioned from about 1 cm to about 95 cm below the soil surface when the subsurface support is inserted under the soil surface.

7. The soil carbon measurement device of claim 1, wherein the ultraviolet light source is a light emitting diode configured to emit a wavelength from 100 nm to 450 nm.

8. The soil carbon measurement device of claim 1, wherein the carbon dioxide sensor is a nondispersive infrared (NDIR) carbon dioxide sensor.

9. The soil carbon measurement device of claim 1, further comprising an above-ground enclosure connected to the subsurface support.

10. The soil carbon measurement device of claim 9, wherein the above-ground enclosure contains a wireless transmitter connected to the carbon dioxide sensor to transmit carbon dioxide concentration measurements.

11. The soil carbon measurement device of claim 1, further comprising a battery connected to supply power to the carbon dioxide sensor.

12. The soil carbon measurement device of claim 1, further comprising a wireless wake-up module configured to turn on the ultraviolet light source and the carbon dioxide sensor upon receiving a wireless signal.

13. A method of measuring soil carbon, comprising:
inserting a soil carbon measurement device at least partially under a soil surface, wherein the soil carbon measurement device comprises an interior volume and a gas-permeable barrier separating the interior volume from soil under the soil surface;
exposing the soil under the soil surface to ultraviolet light; and
measuring a concentration of carbon dioxide in the interior volume of the soil carbon measurement device.

14. The method of claim 13, wherein measuring the concentration of carbon dioxide comprises measuring the concentration during a first time period without exposing the soil to the ultraviolet light and measuring the concentration during a second time period while exposing the soil to the ultraviolet light.

15. The method of claim 14, further comprising measuring the concentration during a third time period after the second period, wherein the soil is not exposed to the ultraviolet light during the third time period.

16. The method of claim 14, further comprising comparing the measured concentration during the first time period with the measured concentration during the second time period.

17. The method of claim 14, wherein the first time period is from about 20 minutes to about 2 hours and wherein the second time period is from about 10 minutes to about 1 hour.

18. The method of claim 13, wherein the soil carbon measurement device comprises an ultraviolet light source and a carbon dioxide sensor.

19. The method of claim 18, further comprising maintaining the ultraviolet light source and the carbon dioxide sensor in a turned-off state and sending a wireless signal to the soil carbon measurement device to wake up the soil carbon measurement device and turn on the ultraviolet light source and the carbon dioxide sensor.

20. A soil carbon monitoring system, comprising:
an array of soil carbon measurement devices distributed over an area of land, the soil carbon measurement devices comprising:
a subsurface support inserted at least partially under a soil surface, wherein the subsurface support comprises an interior volume and a gas-permeable barrier separating the interior volume from soil under the soil surface,
an ultraviolet light source positioned to expose the soil under the soil surface to ultraviolet light,
a carbon dioxide sensor configured to measure a concentration of carbon dioxide in the interior volume of the subsurface support, and
a wireless transmitter configured to transmit the measured concentration of carbon dioxide; and
a monitoring station comprising a wireless receiver configured to the receive measured concentrations of carbon dioxide from the soil carbon measurement devices.
